(19) United States
(12) Patent Application Publication
(10) Pub. No.: US 2025/0261878 A1
(43) Pub. Date: Aug. 21, 2025

(54) Electronics Module and Method

(71) Applicant: Prevayl Innovations Limited, Wilmslow (GB)

(72) Inventors: Gregory William Headley, Wilmslow (GB); Mantas Jurkuvenas, Wilmslow (GB); Michael John Lynch, Cheshire (GB)

(21) Appl. No.: 18/858,391

(22) PCT Filed: Apr. 24, 2023
(86) PCT No.: PCT/GB2023/051072

§ 371 Date: Oct. 21, 2024

(30) Foreign Application Priority Data
Apr. 27, 2022 (GB) 2206095.8
Feb. 7, 2023 (GB) 2301684.3

(51) Int. Cl. A61B 5/11; A61B 5/00; A61B 5/024

(52) U.S. Cl. CPC A61B 5/1118; A61B 5/02416; A61B 5/6802; A61B 2562/0233

(57) ABSTRACT

The electronics module (102) is for monitoring physical activity of a user. The electronics module (102) comprises an electrical sensor (104), an optical sensor (106) and a controller (108). The controller (108) obtains contextual information indicative of the type of wearable article that the electronics module (102) is coupled to or the location of the electronics module on the wearable article. The controller (108) is arranged to use the contextual information to automatically configure the operation of the electrical sensor (104) and the optical sensor 106. In some examples, the controller (108) automatically switches between performing measurements using the electrical sensor (104) and the optical sensor (106) based on the contextual information. The contextual information may indicate whether the electrical sensor (104) is coupled to one or more electrodes of a wearable article.

The present invention is directed towards an electronics module and a method, and in particular towards an electronics module and method for configuring an operation of an electrical sensor and optical sensor of the electronics module.

### BACKGROUND

United Kingdom Patent Application Publication No. GB2596518 A discloses a wearable device for monitoring physical activity of a user. The wearable device is reversibly attachable to a chest strap and an arm strap. The wearable device comprises an ECG sensor arranged to collect ECG measurements only when the wearable device is attached to the user's chest by the chest strap, and a PPG sensor arranged to collect PPG measurements when the wearable device is attached to the user's arm or wrist by the arm strap. The wearable device also comprises control circuitry arranged to switch the wearable device between an ECG only mode and a PPG only mode. In the ECG only mode, the control circuity activates the ECG sensor to collect ECG measurements but prevents the PPG sensor from collecting PPG measurements. In the PPG only mode the reverse is true. A button on the wearable device is arranged to de/activate PPG only mode.

A problem with this approach is that it requires the user to manually switch between the ECG only mode and the PPG only mode by interacting with a button on the wearable device. This can be prone to user error. A button also increases the form factor, cost and complexity of the wearable device.

### SUMMARY

According to the present invention, there is provided an electronics module and method as set out in the appended claims. Other features of the invention will be apparent from the dependent claims, and the description which follows.

According to a first aspect of the disclosure, there is provided an electronics module for monitoring physical activity of a user. The electronics module is arranged to be removably coupled to a wearable article. The electronics module comprises an electrical sensor, an optical sensor, and a controller arranged to obtain contextual information from one or more sensors of the electronics module when the electronics module is coupled to the wearable article, the contextual information being indicative of whether the electrical sensor is connected to an electrode of the wearable article and whether the optical sensor has line of sight with a skin surface of the user. The controller is arranged to use the contextual information to automatically configure the operation of the electrical sensor and the optical sensor. When the contextual information indicates that the electrical sensor is connected to the electrode, the controller is arranged to control the electrical sensor to perform measurements. When the contextual information indicates that the optical sensor has line of sight with a skin surface of the user, the controller is arranged to control the optical sensor to perform measurements.

The contextual information is indicative of whether the electrical sensor is connected to an electrode of the wearable article and whether the optical sensor has line of sight with a skin surface of the user. The optical sensor may have line of sight with a skin surface of the user if the optical sensor is not obscured by the wearable article. The contextual information may be indicative of the type of wearable article the electronics module is coupled to or the location of the electronics module on the wearable article.

Some example wearable articles may comprise one or more electrodes and the electrical sensor may connect to the one or more electrodes when coupled to the wearable article. When coupled to such wearable articles, the controller determines from the contextual information that the electronics module is coupled to a type of wearable article that enables measurement using the electrical sensor. Some example wearable articles may comprise one or more openings to expose the optical sensor of the electronics module and allow the optical sensor to have line of sight with a skin surface of the user when coupled to the wearable article. When coupled to such wearable articles, the controller determines from contextual information that the electronics module is coupled to a type of wearable article that enables measurement using the optical sensor. Different wearable articles may be designed for different purposes. Some wearable articles may be designed for monitoring using the electrical sensor and not the optical sensor while other wearable articles may be desired for monitoring using the optical sensor and not the electrical sensor.

Some wearable articles may comprise multiple locations at which the electronics module may be retained. That is, the electronics module may be arranged to be removably coupled to a single wearable article at a plurality of different locations. When coupled to such a wearable article at a first location, the electrical sensor of the electronics module may be brought into communication with an electrode of the wearable article. The controller may therefore determine that the electronics module is coupled to the wearable article at a location that enables measurement using the electrical sensor. When coupled to a such a wearable article at a second location, the optical sensor may have line of sight with a skin surface of the user. The controller may therefore determine that the electronics module is coupled to the wearable article at a location that enables measurement using the optical sensor.

Advantageously, the operation of the electrical sensor and the optical sensor is automatically configured based on the contextual information. This enables the electronics module to adapt its operation based on the type of wearable article it is coupled to or the location of the electronics module on the wearable article without a user input such as via a button of the electronics module.

The one or more sensors may comprise one or a combination of the electrical sensor, the optical sensor, a motion sensor, a capacitive sensor, an electrostatic sensor, a magnetic field sensor, and a force sensor.

The contextual information may be obtained from a contextual sensor of the electronics module. The contextual information is obtained without user input.

The controller may be arranged to use the contextual information to automatically switch the electronics module between performing measurements using the electrical sensor and the optical sensor.

The controller may be arranged to automatically switch the electronics module between a first measurement mode, in which the electrical sensor is used to perform measurements and the optical sensor is not used to perform measurements, and a second measurement mode, in which the electrical sensor is not used to perform measurements and the optical sensor is used to perform measurements.

Advantageously, the controller is able to use either the electrical sensor or the optical sensor to perform measurements based on the contextual information. This can reduce the power consumption of the electronics module and avoid performing measurements which are not suited to the type of wearable article that the electronics module is coupled to or the location of the electronics module on the wearable article.

As explained above, the contextual information is indicative of whether the electrical sensor is coupled to one or more electrodes of a wearable article.

The controller is arranged to perform measurements using the electrical sensor when the contextual information indicates that the electrical sensor is coupled to one or more electrodes of a wearable article. The controller is arranged to perform measurements using the optical sensor when the contextual information indicates that the electrical sensor is not coupled to one or more electrodes of a wearable article.

As explained above, the contextual information is indicative of whether the optical sensor has line of sight with a skin surface of the user. The controller is arranged to perform measurements using the optical sensor when the contextual information indicates that the optical sensor has line of sight with a skin surface of the user. The controller may be arranged to perform measurements using the electrical sensor when the contextual information does not indicate that the optical sensor has line of sight with a skin surface of the user.

The contextual information may comprise motion data for the electronics module. The motion data may be recorded by a motion sensor of the electronics module.

Advantageously, the contextual information may comprise motion data. A motion sensor may be incorporated into the electronics module to trigger the electronics module from a sleep state (a low power mode) to a wake state (a power mode having a higher power consumption than the sleep state). The motion sensor may also be used to track the motion of the wearer during activities. Using the motion sensor as the source of the contextual information reuses what may be an existing component of the electronics module which simplifies the construction of the electronics module and avoids increasing the form factor and cost of the electronics module.

The motion data may identify the orientation of the electronics module.

The motion data may indicate that the electrical sensor is connected to an electrode of the wearable article when the motion data identifies the electronics module as being in a first orientation. The motion data identifying that the electronics module is in the first orientation may also indicate that the optical sensor is obscured by the wearable article and does not have line of sight with a skin surface of the user. The motion data may therefore indicate that the electronics module is coupled to a first type of wearable article, or a first location on a wearable article, that enables the electrical sensor to perform measurements using an electrode of the wearable article.

The motion data may indicate that the optical sensor has line of sight with a skin surface of the user when the motion data identifies the electronics module as being in a second orientation different to the first orientation. The motion data identifying the electronics module as being in the second orientation may also indicate that the electrical sensor is not connected to an electrode of the wearable article. The motion data may therefore indicate that the electronics module is coupled to a second type of wearable article, or a second location on a wearable article, that enables the optical sensor to perform measurements.

The contextual information may comprise an electrical measurement performed by the electrical sensor.

Advantageously, the electrical sensor may be used as the source of the contextual information. This reuses an existing component of the electronics module which simplifies the construction of the electronics module and avoids increasing the form factor and cost of the electronics module.

The electrical measurement may indicate that the electrical sensor is connected to an electrode of the wearable article when the electrical measurement has a first value. The electrical measurement having a first value may also indicate that the optical sensor is obscured by the wearable article and does not have line of sight with a skin surface of the user. The electrical measurement may therefore indicate that the electronics module is coupled to a first type of wearable article, or a first location on a wearable article, that enables the electrical sensor to perform measurements using an electrode.

The electrical measurement may indicate that the optical sensor has line of sight with a skin surface of the user when the electrical measurement has a second value different to the first value. The electrical measurement having a second value may also indicate that the electrical sensor is not connected to an electrode of the wearable article. The electrical measurement may therefore indicate that the electronics module is coupled to a second type of wearable article, or a second location on a wearable article, that enables the optical sensor to perform measurements.

The electrical measurement may indicate that the electrical sensor is connected to an electrode of the wearable article when the electrical measurement has a signal quality above a threshold value. The electrical measurement having a signal quality above a threshold value may also indicate that the optical sensor is obscured by the wearable article and does not have line of sight with a skin surface of the user. The electrical measurement may therefore indicate that the electronics module is coupled to a first type of wearable article, or a first location on a wearable article, that enables the electrical sensor to perform measurements using an electrode.

The electrical measurement may indicate that the optical sensor has line of sight with a skin surface of the user when the electrical measurement has a signal quality below the threshold value. The electrical measurement having a signal quality below a threshold value may also indicate that the electrical sensor is not connected to an electrode of the wearable article. The electrical measurement may therefore indicate that the electronics module is coupled to a second type of wearable article, or a second location on a wearable article, that enables the optical sensor to perform measurements.

The contextual information may comprise capacitance data measured by a capacitive sensor of the electronics module.

Advantageously, the contextual information may comprise capacitance data. A capacitive sensor may be incorporated into the electronics module to trigger the electronics module from a sleep state (a low power mode) to a wake state (a power mode having a higher power consumption than the sleep state). Using the capacitive sensor as the source of the contextual information reuses what may be an existing component of the electronics module which simplifies the construction of the electronics module and avoids increasing the form factor and cost of the electronics module.

The capacitance data may indicate that the electrical sensor is connected to an electrode of the wearable article when the capacitance data has a first value. The capacitance data having the first value may also indicate that the optical sensor is obscured by the wearable article and does not have line of sight with a skin surface of the user. The capacitance data may therefore indicate that the electronics module is coupled to a first type of wearable article, or a first location on a wearable article, that enables the electrical sensor to perform measurements.

The capacitance data may indicate that the optical sensor has line of sight with a skin surface of a user when the capacitance data has a second value different to the first value. The capacitance data having the second value may also indicate that the electrical sensor is not connected to an electrode of the wearable article. The capacitance data may therefore indicate that the electronics module is coupled to a second type of wearable article, or a second location on a wearable article, that enables the optical sensor to perform measurements.

The contextual information may comprise electrostatic data measured by an electrostatic sensor of the electronics module.

The electrostatic data may indicate that the electrical sensor is connected to an electrode of the wearable article when the electrostatic data has a first value. The electrostatic data having the first value may also indicate that the optical sensor is obscured by the wearable article and does not have line of sight with a skin surface of the user. The electrostatic data may therefore indicate that the electronics module is coupled to a first type of wearable article, or a first location on a wearable article, that enables the electrical sensor to perform measurements.

The electrostatic data may indicate that the optical sensor has line of sight with a skin surface of the user when the electrostatic data has a second value different to the first value. The electrostatic data having the second value may also indicate that the electrical sensor is not connected to an electrode of the wearable article. The electrostatic data may therefore indicate that the electronics module is coupled to a second type of wearable article, or a second location on a wearable article, that enables the optical sensor to perform measurements.

The contextual information may comprise force data indicative of a force applied to the electronics module.

The force data may indicate that the electrical sensor is connected to an electrode of the wearable article when the force data has a first value. The force data having the first value may also indicate that the optical sensor is obscured by the wearable article and does not have line of sight with a skin surface of the user. The force data may therefore indicate that the electronics module is coupled to a first type of wearable article, or a first location on a wearable article, that enables the electrical sensor to perform measurements.

The force data may indicate that the optical sensor has line of sight with a skin surface of the user when the force data has a second value different to the first value. The force data having the second value may also indicate that the electrical sensor is not connected to an electrode of the wearable article. The force data may therefore indicate that the electronics module is coupled to a second type of wearable article, or a second location on a wearable article, that enables the optical sensor to perform measurements.

The contextual information may comprise an optical measurement performed by the optical sensor.

Advantageously, the optical sensor may be used as the source of the contextual information. This reuses an existing component of the electronics module which simplifies the construction of the electronics module and avoids increasing the form factor and cost of the electronics module.

The optical measurement may indicate that the electrical sensor is connected to an electrode of the wearable article when the optical measurement has a first value. The optical measurement having the first value may also indicate that the optical sensor is obscured by the wearable article and does not have line of sight with a skin surface of the user. The optical measurement may therefore indicate that the electronics module is coupled to a first type of wearable article, or a first location on a wearable article, that enables the electrical sensor to perform measurements.

The optical measurement may indicate that the optical sensor has line of sight with a skin surface of the user when the optical measurement has a second value different to the first value. The optical measurement having the second value may also indicate that the electrical sensor is not connected to an electrode of the wearable article. The optical measurement may therefore indicate that the electronics module is coupled to a second type of wearable article, or a second location on a wearable article, that enables the optical sensor to perform measurements.

The contextual information may comprise magnetic field data measured by a magnetic field sensor of the electronics module.

The magnetic field data may indicate that the electrical sensor is connected to an electrode of the wearable article when the magnetic field data has a first value. The magnetic field data having the first value may also indicate that the optical sensor is obscured by the wearable article and does not have line of sight with a skin surface of the user. The magnetic field measurement may therefore indicate that the electronics module is coupled to a first type of wearable article, or a first location on a wearable article, that enables the electrical sensor to perform measurements.

The magnetic field data may indicate that the optical sensor has line of sight with a skin surface of the user when the magnetic field data has a second value different to the first value. The magnetic field data having the second value may also indicate that the electrical sensor is not connected to an electrode of the wearable article. The magnetic field data may therefore indicate that the electronics module is coupled to a second type of wearable article, or a second location on a wearable article, that enables the optical sensor to perform measurements.

The contextual information may comprise any combination of motion data, an electrical measurement, capacitance data, electrostatic data, force data, an optical measurement and magnetic field data.

The electrical sensor may comprise a bioelectrical and/or bioimpedance sensor.

The optical sensor may comprise a PPG sensor.

According to a second aspect of the disclosure, there is provided a method performed by an electronics module for monitoring physical activity of a user, the method comprising obtaining contextual information from one or more sensors of the electronics module when the electronics module is coupled to the wearable article, the contextual information is indicative of whether the electrical sensor is connected to an electrode of the wearable article and whether the optical sensor has line of sight with a skin surface of the user, and using the contextual information to automatically configure the operation of an electrical sensor and an optical sensor of the electronics module. When the contextual information indicates that the electrical sensor is connected to the electrode, the method comprises controlling the electrical sensor to perform measurements. When the contextual information indicates that the optical sensor has line of sight with a skin surface of the user, the method comprises controlling the optical sensor to perform measurements.

According to a third aspect of the disclosure, there is provided a method performed by an electronics module for monitoring physical activity of a user, the method comprising coupling the electronics module to a first type of wearable article or a first location on a wearable article, obtaining contextual information from one or more sensors of the electronics module when the electronics module is coupled to the first type of wearable article or first location on the wearable article, the contextual information is indicative of whether the electrical sensor is connected to an electrode of the wearable article and whether the optical sensor has line of sight with a skin surface of the user, using the contextual information to automatically configure the operation of an electrical sensor and an optical sensor of the electronics module, coupling the electronics module to a second type of wearable article or a second location on the wearable article, obtaining contextual information from one or more sensors of the electronics module when the electronics module is coupled to the second type of wearable article or second location on the wearable article, the contextual information is indicative of whether the electrical sensor is connected to an electrode of the wearable article and whether the optical sensor has line of sight with a skin surface of the user, and using the contextual information to automatically configure the operation of the electrical sensor and the optical sensor. When the contextual information indicates that the electrical sensor is connected to the electrode, the method comprises controlling the electrical sensor to perform measurements. When the contextual information indicates that the optical sensor has line of sight with a skin surface of the user, the method comprises controlling the optical sensor to perform measurements.

According to a fourth aspect of the disclosure, there is provided a system comprising an electronics module of the first aspect of the disclosure and a wearable article.

The system may comprise a first type of wearable article and a second type of wearable article.

The wearable article may comprise an electronics module holder arranged to removably couple the electronics module to the wearable article.

The wearable article may be arranged to receive the electronics module at a plurality of locations. At a first location, the electronics module may be brought into communication with an electrode of the wearable article. At a second location, the optical sensor may have line of sight with a skin surface of the user.

According to a fifth aspect of the disclosure, there is provided a system comprising an electronics module and a wearable article. The electronics module comprises an electrical sensor, an optical sensor and a controller arranged to control the operation of the electrical sensor and the optical sensor. The wearable article comprises an electrode, a first electronics module holder arranged to removably retain the electronics module, and a second electronics module holder arranged to removably retain the electronics module. When retained by the first electronics module holder, the electrical sensor is able to be brought into communication with the electrode. When retained by the second electronics module holder, the optical sensor has line of sight through an opening of the wearable article.

Advantageously, the electronics module may be received at multiple locations on a single wearable article. The first electronics module holder and second electronics module holder may be a different locations on the wearable article. The electronics module may be swapped between the first electronics module holder and the second electronics module holder depending on the type of activity being performed or the desired metric to be measured. Alternatively, multiple electronics modules may be used simultaneously with a single wearable article to obtain a greater volume of measurements and measurements from different locations on the body.

The wearable article may comprise a plurality of electrodes. The electrical sensor may be brought into communication with the plurality of electrodes when retained by the first electronics module holder.

The controller may configure the operation of the electrical sensor and the optical sensor dependent on whether the electronics module is received by the first electronics module holder or the second electronics module holder.

The controller may use contextual information as per the first aspect of the disclosure. The electronics module may be the electronics module as described above in relation to the first aspect of the disclosure. In some examples, the operation of the electrical sensor and optical sensor is manually triggered by a user interacting with a user interface element such as a button of the electronics module.

The wearable article may comprise more than two electronics module holders.

According to a sixth aspect of the disclosure, there is provided a wearable article comprising an electrode, a first electronics module holder arranged to removably retain an electronics module in a first orientation, and a second electronics module holder arranged to removably retain the electronics module in a second orientation different to the first orientation. When retained by the first electronics module holder, the electrical sensor is able to be brought into communication with the electrode. When retained by the second electronics module holder, the optical sensor has line of sight through an opening of the wearable article.

According to a seventh aspect of the disclosure, there is provided an electronics module for monitoring physical activity of a user, the electronics module is arranged to be removably coupled to any of a single wearable article at a plurality of locations or a plurality of different wearable articles. The electronics module comprises an optical sensor arranged to perform optical measurements of the user depending on the particular location at which the electronics module is coupled to the single wearable article or the particular wearable article of the plurality of different wearable articles the electronics module is coupled to. The electronics module comprises an electrical sensor arranged to perform electrical measurements of the user depending on the particular location at which the electronics module is coupled to the single wearable article or the particular wearable article of the plurality of different wearable articles the electronics module is coupled to. The electronics module comprises a controller arranged to obtain contextual information from one or more sensors of the electronics module when the electronics module couples to the single wearable article at one of the plurality of locations or couples to one of the plurality of different wearable articles. The contextual information is indicative of whether the electrical sensor is connected to an electrode of the wearable article and whether the optical sensor has line of sight with a skin surface of the user. When the contextual information indicates that the electrical sensor is connected to the electrode, the controller is arranged to control the electrical sensor to perform measurements. When the contextual information indicates that the optical sensor has line of sight with a skin surface of the user, the controller is arranged to control the optical sensor to perform measurements.

According to a eighth aspect of the disclosure, there is provided an electronics module for monitoring physical activity of a user, the electronics module comprising: an electrical sensor; an optical sensor; a contextual sensor; and a controller arranged to obtain contextual information from the contextual sensor when the electronics module is coupled to the wearable article, and automatically configure the operation of the electrical sensor and the optical sensor dependent on the contextual information.

The contextual sensor may comprise a motion sensor. The controller may be arranged to obtain motion data from the motion sensor, and automatically configure the operation of the electrical sensor and the optical sensor dependent on whether the motion data identifies the electronics module as being in a first orientation or a second orientation different to the first orientation.

### BRIEF DESCRIPTION OF THE DRAWINGS

To easily identify the discussion of any particular element or act, the most significant digit or digits in a reference number refer to the figure number in which that element is first introduced.

FIG. 1 illustrates an example electronics module according to aspects of the present disclosure.

FIG. 2 illustrates the electronics module of FIG. 1 coupled to a first type of wearable article.

FIG. 3 illustrates the electronics module of FIG. 1 coupled to a second type of wearable article.

FIG. 4 illustrates the electronics module of FIG. 1 coupled to a third type of wearable article.

FIG. 5 illustrates the electronics module of FIG. 1 coupled to the first type of wearable article.

FIG. 6 illustrates the electronics module of FIG. 1 coupled to the second type of wearable article.

FIG. 7A illustrates another example wearable article according to aspects of the present disclosure.

FIG. 7B illustrates the electronics module of FIG. 1 coupled to the wearable article of FIG. 7A.

FIG. 8 illustrates a schematic of an example electronics module in accordance with aspects of the present disclosure.

FIG. 9 illustrates a method for monitoring physical activity of a user in accordance with aspects of the present disclosure.

FIG. 10A and FIG. 10B illustrate front and side views of an example wearable article according to aspects of the present disclosure.

FIG. 11A and FIG. 11B illustrate front and side views of an example bra according to aspects of the present disclosure.

FIG. 12 illustrates a view of the bottom surface of an example electronics module according to aspects of the present disclosure.

FIG. 13 illustrates the electronics module of FIG. 12 with one of the contacts removed.

FIG. 14 illustrates the electronics module of FIG. 12 coupled to an example wearable article according to aspects of the present disclosure.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings but are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise.

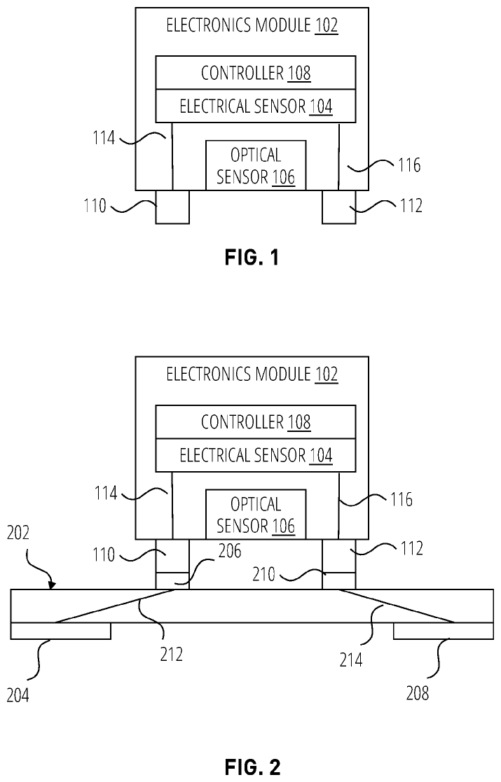

FIG. 1 shows an example electronics module 102 for monitoring physical activity of a user according to aspects of the present disclosure.

The electronics module 102 is arranged to be worn by a user.

The electronics module 102 is typically arranged to be removably coupled to a wearable article. The electronics module 102 can be removed from the wearable article so that the wearable article can be washed without damaging the internal electronics of the electronics module 102. The electronics module 102 can also be removed from the wearable article for charging. In addition, the electronics module 102 can be used with a number of different types of wearable article. The electronics module 102 can be removed from one wearable article and coupled to another wearable article. The electronics module 102 can be coupled to a single wearable article at multiple different locations.

Generally, the electronics module 102 comprises all of the components required for data transmission and processing. In this way, the manufacture of the wearable article may be simplified. In addition, it may be easier to clean a wearable article which has fewer electronic components attached thereto or incorporated therein. Furthermore, the removable electronics module 102 may be easier to maintain or troubleshoot than embedded electronics.

The wearable article typically comprises an electronics module holder arranged to removably retain the electronics module 102. The electronics module holder enables the electronics module 102 to be attached to and removed from the wearable article.

The electronics module holder may be in the form of a mechanical interface such as a clip, a plug and socket arrangement, or a frame. The mechanical interface may be configured to maintain the electronics module 102 in a particular orientation with respect to the wearable article when the electronics module 102 is coupled to the wearable article. This may be beneficial in ensuring that the electronics module 102 is securely held in place with respect to the wearable article. The mechanical coupling may be maintained using friction or a positively engaging mechanism, for example.

The electronics module holder may comprise a pocket sized to receive and temporarily retain the electronics module 102.

The electronics module holder may comprise a magnetic material that cooperates with a magnetic material of the electronics module 102 to form a magnetic coupling.

The wearable article may be any form of article which may be worn by a user such as a smart watch, necklace, garment, bracelet, or glasses. The wearable article may be a textile article. The wearable article may be a garment. The garment may refer to an item of clothing or apparel. The garment may be a top. The top may be a shirt, t-shirt, blouse, sweater, jacket/coat, or vest. The garment may be a dress, garment brassiere, shorts, pants, arm or leg sleeve, vest, jacket/coat, glove, armband, underwear, headband, hat/cap, collar, wristband, armband, chestband, waistband, stocking, sock, or shoe, athletic clothing, personal protective equipment, including hard hats, swimwear, wetsuit or dry suit.

The wearable article (e.g., a garment) may be constructed from a woven or a non-woven material. The wearable article may be constructed from natural fibres, synthetic fibres, or a natural fibre blended with one or more other materials which can be natural or synthetic. The yarn may be cotton. The cotton may be blended with polyester and/or viscose and/or polyamide according to the application. Silk may also be used as the natural fibre. Cellulose, wool, hemp and jute are also natural fibres that may be used in the wearable article. Polyester, polycotton, nylon and viscose are synthetic fibres that may be used in the wearable article.

The garment may be a tight-fitting garment or a loose-fitting (e.g. freeform garment). A tight-fitting garment helps ensure that the electrodes of the garment are held in contact with or in the proximity of a skin surface of the wearer. The tight-fitting garment may be a compression garment. The tight-fitting garment may be an athletic garment such as an elastomeric athletic garment. A loose-fitting garment is generally more comfortable to wear over extended time periods and during sleep.

The electronics module 102 comprises an electrical sensor 104. The electrical sensor may comprise a biopotential sensor such as an ECG sensor. Biopotential sensors measure the potential across the skin surface. The electrical sensor may comprise a bioimpedance sensor. Bioimpedance sensors typically comprises a source of current and a receiver. The bioimpedance sensor can measure the opposition to electric current through a part of the body of the user. The bioimpedance sensor typically measures electrical resistance and/or reactance. Example bioimpedance sensors include impedance plethysmography sensors which can be used to measure the breathing activity of the user.

In some examples, the electrical sensor 104 is a combined biopotential and bioimpedance sensor.

The electrical sensor 104 may comprise an analogue-to-digital front end for converting analogue signals sensed by the sensor to digital signal values. ADC IC chips for ECG and bioimpedance applications include, for example, the MAX30001 chip produced by Maxim Integrated Products Inc.

The electrical sensor 104 comprises first contact 110 and second contact 112 that are positioned externally on the electronics module 102 and electrically coupled to the remainder of the electrical sensor 104 (e.g., the analogue-to-digital front end) by first conductors 114 and second conductor 116.

The first contact 110 and second contact 112 may directly contact the skin to perform an electrical measurement such as an ECG measurement or bioimpedance measurement. The first contact 110 and second contact 112 may couple to electrodes incorporated into the wearable article to perform the electrical measurement.

The electronics module 102 further comprises an optical sensor 106. The optical sensor 106 may measure the amount of ultraviolet, visible, and/or infrared light in the environment. The optical sensor 106 may comprise a photoplethysmography (PPG) sensor. PPG sensors measure blood volume changes within the microvascular bed of the wearer's tissue. PPG sensors use a light source to illuminate the tissue. Photodetectors within the PPG sensor measure the variations in the intensity of absorbed or reflected light when blood perfusion varies.

The optical sensor 106 may comprise one or more light emitters and one or more photodetectors. The light emitters emit light to the skin tissue of the wearer. The light emitters may emit light in one or more of the infrared, visible, and ultraviolet spectrums. The photodetectors measure the reflected light from the tissue. The photodetectors may measure light in one or more of the infrared, visible and ultraviolet spectrums. Light is more strongly absorbed by blood than surrounding tissues, so the change in blood flow is measurable based upon the change in the intensity of the reflected light over time. This can be used to obtain a heartrate of the wearer. Other properties of the wearer can be determined from the optical sensor 106. The optical sensor 106 is not limited to the use in determining the heartrate of the wearer. The optical sensor 106 may be arranged to measure the oxygen saturation of the wearer. Oxygen saturation is the fraction of oxygen-saturated haemoglobin relative to total haemoglobin (unsaturated +saturated) in the blood. The optical sensor 106 may be arranged to measure the capillary perfusion of the wearer. The optical sensor 106 may be useable to measure the capillary perfusion using a double-wavelength method. The capillary perfusion can be derived from a variation in the detected signal strength. The optical sensor 106 may be arranged to measure the temperature of the wearer.

The optical sensor 106 is positioned proximate to a bottom surface of the electronics module 102 that faces towards the skin surface of the wearer when the electronics module 102 is worn. The optical sensor 106 may be positioned inside a housing of the electronics module 102. The housing may have an opening to allow for the optical sensor 106 to have line of sight out of the electronics module 102. The housing may comprise a window that covers the optical sensor 106 to protect against ingress of water, dust and other debris into the housing. The window may be formed from an optically transparent or translucent material.

The first contact 110 and the second contact 112 are located externally outside of the housing of the electronics modules 102 and in this example are positioned on an external side of the bottom surface of the housing. The first conductor 114 and second conductor 116 extend through the housing to connect the first contact 110 and the second contact 112 to the remainder of the electrical sensor 104 located inside the housing. The first contact 110 and the second contact 112 are not required to be located on an external side of the bottom surface of the housing in all examples and may for example be provided on a side surface of the housing.

The electronics module 102 further comprises a controller 108. The controller 108 is communicatively coupled to the electrical sensor 104 and the optical sensor 106 and is arranged to receive measurement data from the electrical sensor 104 and the optical sensor 106 and control the operation of the electrical sensor 104 and the optical sensor 106.

FIG. 2 shows an example assembly formed by the electronics module 102 of FIG. 1 and a wearable article 202 according to aspects of the present disclosure. The electronics module 102 is removably coupled to the wearable article 202.

The wearable article 202 comprises a first electrode 204 and a second electrode 208. The first electrode 204 and the second electrode 208 are arranged such that they come into contact with or come into close proximity with a skin surface of the wearer when the wearable article 202 is worn.

The electrodes may be made of a (electrically) conductive material such as a conductive yarn, conductive ink, conductive transfer, or conductive paste. When formed from conductive yarn, the electrodes may be knitted, woven, embroidered, stitched or otherwise incorporated into the wearable article 202. The electrodes may be integrally formed with the wearable article 202 such as by being integrally knitted with the wearable article 202.

The first electrode 204 is communicatively coupled to a first connection region 206 of the wearable article 202 via first conductive pathway 212. The second electrode 208 is communicatively coupled to a second connection region 210 of the wearable article 202 via second conductive pathway 214.

The first contact 110 of the electronics module 102 contacts the first connection region 206 such that the electrical sensor 104 is electrically coupled to the first electrode 204. The second contact 112 of the electronics module 102 contacts the second connection region 210 such that the electrical sensor 104 is electrically coupled to the second electrode 208.

In this example, the optical sensor 106 does not have line of sight with the skin surface of the wearer as the wearable article 202 forms a barrier between the optical sensor and the skin surface.

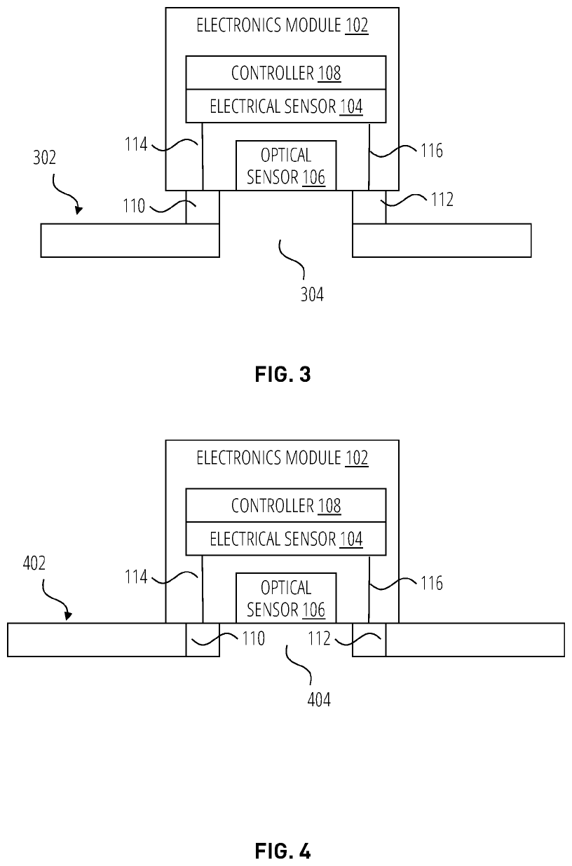

FIG. 3 shows another example assembly formed by the electronics module 102 of FIG. 1 and a wearable article 302 according to aspects of the present disclosure. The electronics module 102 is removably coupled to the wearable article 302.

In this example, the wearable article 302 comprises an opening 304 positioned such that when the electronics module 102 is coupled to the wearable article 302 the optical sensor 106 has line of sight through the opening 304 and can thus perform a measurement from the skin surface of the wearer. The opening 304 may be a hole formed in a fabric of the wearable article 302. The opening 304 may be covered by a window. The window may be formed from an optically transparent or translucent material. The optical sensor 106 may extend through the opening 304 or may extend at least partially into the opening 304, but this is not required in all examples.

In this example, the wearable article 302 does not comprise electrodes, connection regions, or conductive pathways. The first contact 110 and the second contact 112 rest on the material of the wearable article 302 (e.g., a fabric) and are not able to perform a measurement from the skin surface of the wearer.

The wearable article 302 may be a wrist strap, arm strap/sleeve or leg strap/sleeve. For such wearable articles, it may not be practical to incorporate electrodes into the wearable article and/or the measurement accuracy of an electrical sensor using such electrodes may be limited.

The wearable article 302 may be an item of clothing such as a top, bottoms, or underwear. The wearable article 302 may be intended to be worn over extended time periods such as at night while the wearer is sleeping. For such wearable articles, it may be undesirable to incorporate electrodes as they may cause user discomfort over extended time periods. This is particularly the case if a high degree of compression is desired to hold the electrodes in contact with the skin surface.

The wearable article 302 may also not include electrodes to reduce the cost of the wearable article 302 or improve the longevity of the wearable article 302. The longevity may refer to the number of times the wearable article 302 may be laundered without degrading the performance of the wearable article 302.

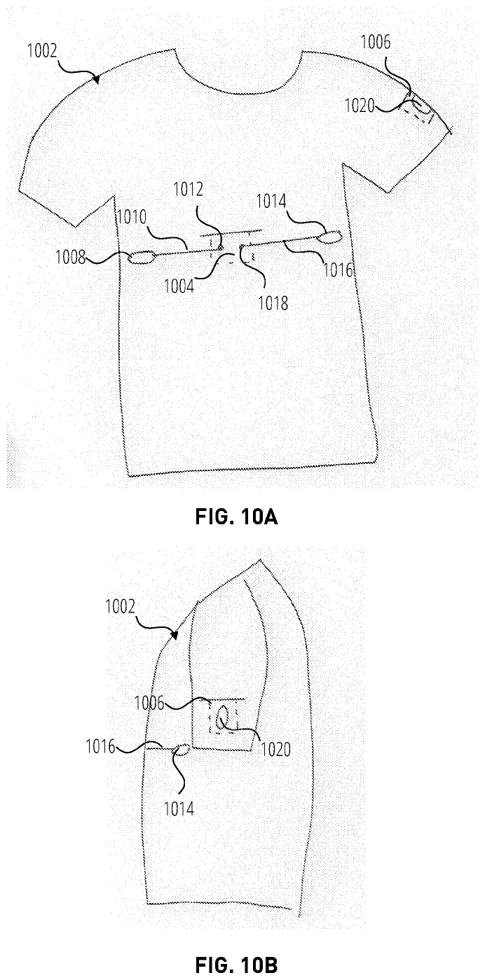

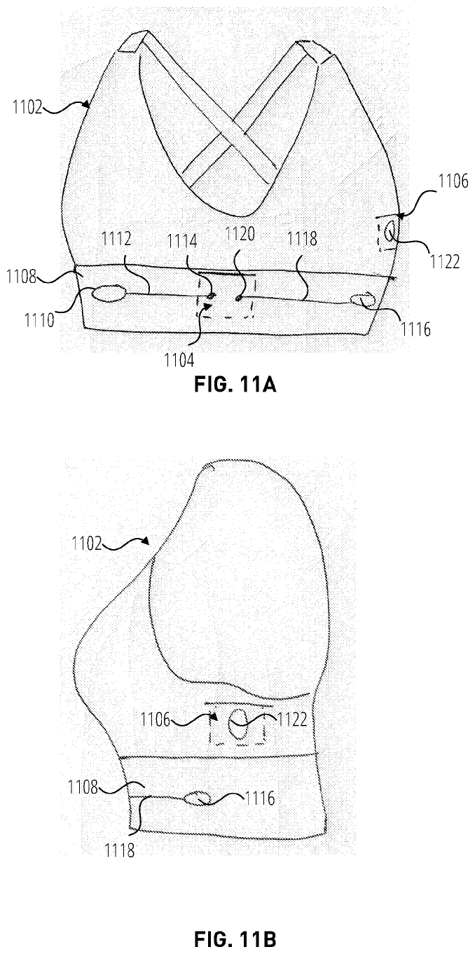

In other examples, the features of the wearable article 202 and wearable article 302 may be combined in a single wearable article. The wearable article may comprise a first electronics module holder and a second electronics module holder. When retained by the first electronics module holder, the electrical sensor 104 is brought into communication with the first electrode 204 and second electrode 208 via the conductive pathways 212, 214 and connection regions 206, 210. When retained by the second electronics module holder, the optical sensor 106 has line of sight through the opening 304 of the wearable article. This is described in greater detail in relation to FIG. 10A to FIG. 11B.

FIG. 4 shows another example assembly formed by the electronics module 102 of FIG. 1 and a wearable article 402 according to aspects of the present disclosure.

The electronics module 102 is removably coupled to the wearable article 402 such that it is retained by the wearable article 202 when worn.

In this example, the wearable article 402 comprises an opening 404 positioned such that when the electronics module 102 is coupled to the wearable article 402 the optical sensor 106 has line of sight through the opening 404 and can thus perform a measurement from the skin surface of the wearer. The opening 404 may be a hole formed in a fabric of the wearable article 402. The opening 404 may be covered by a window. The window may be formed from an optically transparent or translucent material. The optical sensor 106 may extend through the opening 404 or may extend at least partially into the opening 304, but this is not required in all examples.

In this example, the wearable article 402 does not comprise electrodes, connection regions, or conductive pathways. The first contact 110 and the second contact 112 extend through the opening 404 of the wearable article 402 (e.g., a fabric) and are able to contact or come into close proximity with a skin surface of the wearer.

In this example, the electrical sensor 104 is able to perform electrical measurements directly from the skin surface rather than via electrodes of the wearable article.

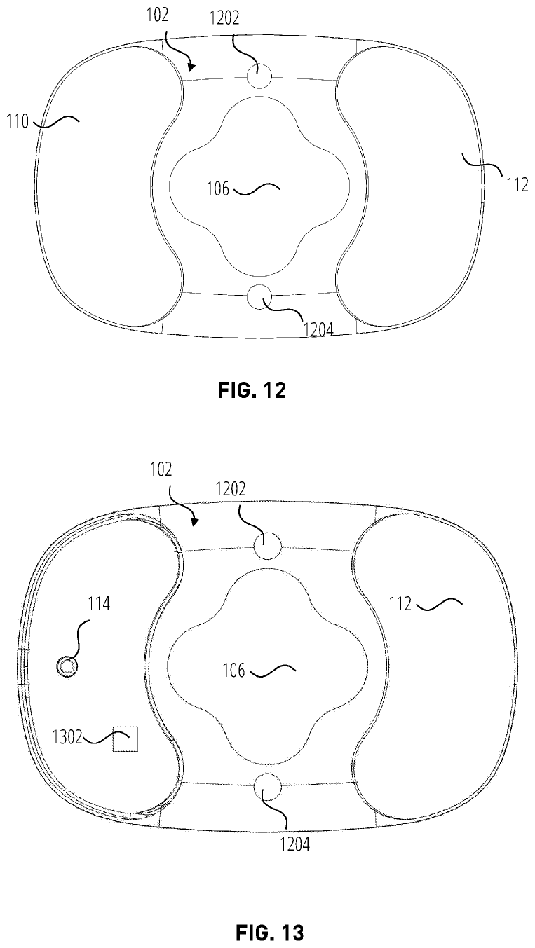

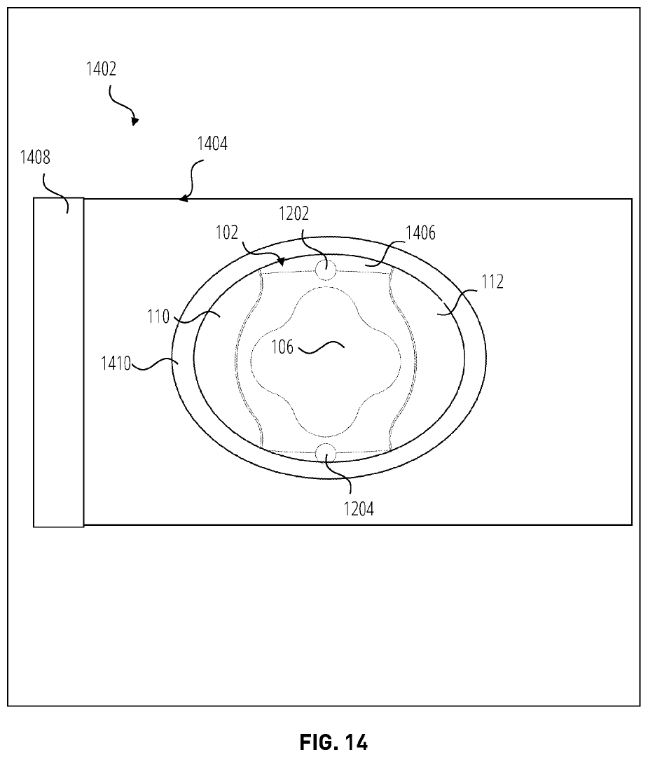

It is not required that both the first contact 110 and the second contact 112 are exposed by the opening 404 or are fully exposed by the opening 404. Instead, one or both of the first contact 110 and the second contact 112 may be partially or fully exposed by the opening 404 such that they are able to contact or come into close proximity with a skin surface of the wearer. In some examples, the electrical sensor 104 is not required to perform electrical measurements directly from the skin surface. Instead, the exposed contact 110, 112 may acts a coupling medium to thermally couple a hidden temperature sensor to the skin surface. This is described in greater detail below in relation to FIG. 12, FIG. 13, and FIG. 14.

The wearable article 402 may be an item of clothing such as a top, bottoms, or underwear. The wearable article 402 may be intended to be worn over extended time periods such as at night while the wearer is sleeping. For such wearable articles, it may be undesirable to incorporate electrodes as they may cause user discomfort over extended time periods. This is particularly the case if a high degree of compression is desired to hold the electrodes in contact with the skin surface.

The wearable article 402 may also not include electrodes to reduce the cost of the wearable article 302 or improve the longevity of the wearable article 402. The longevity may refer to the number of times the wearable article 402 may be laundered without degrading the performance of the wearable article 402.

In other examples, the features of the wearable article 202 and wearable article 402 may be combined in a single wearable article. The wearable article may comprise a first electronics module holder and a second electronics module holder. When retained by the first electronics module holder, the electrical sensor 104 is brought into communication with the first electrode 204 and second electrode 208 via the conductive pathways 212, 214 and connection regions 206, 210. When retained by the second electronics module holder, the optical sensor 106 has line of sight through the opening 404 of the wearable article. This is described in greater detail in relation to FIG. 10A to FIG. 11B.

The electronics module 102 is arranged to be coupled to different types of wearable article. The different types of wearable articles may comprise wrist straps, arm bands, arm sleeves, leg sleeves, and clothing (e.g., tops, bottoms, underwear).

A first type of wearable article may comprise electrodes that couple to the electrical sensor 104 of the electronics module 102. Some of the first type of wearable article may also comprise openings to allow for the optical sensor 106 to measure properties from the skin surface while others, such as shown in FIG. 2, do not comprise openings such that the optical sensor 106 is unable to perform a measurement from the skin surface.

The first type of wearable article may be intended to be used for high quality monitoring of the wearer. High quality refers to the measurements being accurate and reliable. Generally, higher accuracy monitoring is obtained using electrodes (e.g., ECG monitoring) as compared to using the optical sensor along (e.g. PPG monitoring). The first type of wearable article may be an item of sportswear or performance wear or a wearable article intended for use in an industrial (e.g., construction), military or healthcare setting.

A second type of wearable article may not comprise electrodes that couple to the electrical sensor 104 of the electronics module 102. The second type of wearable article may comprise openings to allow for the optical sensor 106 to measure properties from the skin surface. The first and second electrical contacts may rest on the wearable article as show in FIG. 3 such that the electrical sensor 104 is unable to perform a measurement from the skin surface or may extend through the opening of the wearable article as shown in FIG. 4 such that the electrical sensor 104 is able to perform measurements directly from the skin surface.

The second type of wearable article may be intended to be used when high quality monitoring of the wearer is not required. The second type of wearable article may be an item of everyday wearer such as leisure wear or nightwear. The second type of wearable article may be a wrist strap, arm band/sleeve or leg band/sleeve.

It is desirable to configure the operation of the electrical sensor 104 and the optical sensor 106 dependent on the type of wearable article that the electronics module 102 is coupled to.

In some examples, it may be desirable to perform measurements using the electrical sensor 104 and not perform measurements using the optical sensor 106. This may occur when the electronics module 102 is coupled to a wearable article that includes electrodes but does not comprise an opening for the optical sensor 106 as per FIG. 2.

In some examples, it may be desirable to perform measurements using the electrical sensor 104 and the optical sensor 106. This may occur when the electronics module 102 is coupled to a wearable article that does not include electrodes and an opening for the optical sensor 106. This may occur when the electronics module 102 is coupled to a wearable article 202 with an opening for the optical sensor 106 and that enables the first contact 110 and the second contact 112 to contact with or be positioned in close proximity to the skin surface as per FIG. 4.

In some examples, it may be desirable to perform using measurements using the optical sensor 106 and not perform measurements using the electrical sensor 104. This may occur when the electronics module 102 is coupled to a wearable article that includes an opening for the optical sensor 106, does not comprise electrodes, and does not enable the first contact 110 and the second contact 112 to be positioned in contact with or in close proximity to the skin surface as per FIG. 3.

The controller 108 of the electronics module 102 obtains contextual information indicative of the type of wearable article that the electronics module 102 is coupled to or the particular location of the electronics module 102 on a wearable article. In other words, the contextual information is indicative of whether the electrical sensor is connected to an electrode of the wearable article and whether the optical sensor has line of sight with a skin surface of the user. Using the contextual information, the controller 108 automatically, without user input, configures the operation of the electrical sensor 104 and the optical sensor 106.

This may involve the controller 108 switching between performing measurements using the electrical sensor 104, performing measurements using the optical sensor 106 and performing measurements using the electrical sensor 104 and the optical sensor 106. The measurements performed by the electrical sensor 104 and/or the optical sensor 106 may be configured using the contextual information. For example, a first signal processing algorithm may be used for the electrical sensor 104 when coupled to a wearable article with electrodes and a second signal processing algorithm may be used for the electrical sensor 104 when coupled to a wearable article which enables the first contact 110 and the second contact 112 to directly contact or be positioned in close proximity with the skin surface.

The contextual information may be obtained from a sensor of the electronics module 102. The sensor may be referred to as a contextual sensor. The contextual sensor measures a property of the electronics module 102 which is indicative of the type of wearable article that the electronics module 102 is coupled to or the location of the electronics module 102 on the wearable article. In other words, the contextual sensor measures a property of the electronics module 102 that indicates whether the electrical sensor is connected to an electrode of the wearable article and whether the optical sensor has line of sight with a skin surface of the user.

In some examples, the contextual information comprises motion data for the electronics module 102. The contextual sensor comprises/is a motion sensor of the electronics module 102. The motion sensor may comprise one or more of an accelerometer, a gyroscope and a magnetometer. The motion sensor may be in the form of an inertial measurement unit.

The motion data may identify the orientation of the electronics module 102. The motion sensor may comprise a gyroscope in this example. The orientation of the electronics module 102 may identify the type of wearable article that the electronics module 102 is coupled or the location of the electronics module 102 on the wearable article. In other words, the orientation of the electronics module 102 indicates whether the electrical sensor is connected to an electrode of the wearable article and whether the optical sensor has line of sight with a skin surface of the user.

In examples, the electronics module 102 may be retained in different orientations for different wearable articles. The wearable article may comprise an electronics module holder such as a pocket to retain the electronics module 102. The electronics module holder may be configured differently for different types of wearable article so as to hold the electronics module 102 in different orientations. Likewise, the electronics module 102 may be retained in different orientations at different locations on a single wearable article. The wearable article may comprise a plurality of electronics module holders, such as pockets, which hold the electronics module in different orientations. The controller 108 can configure the electrical sensor 104 and the optical sensor 106 in a first way when the motion data identifies the electronics module 102 as being coupled to the wearable article in a first orientation and can configure the electrical sensor 104 and the optical sensor 106 in a second way when the motion data identifies the electronics module 102 as being coupled to the wearable article in a second orientation.

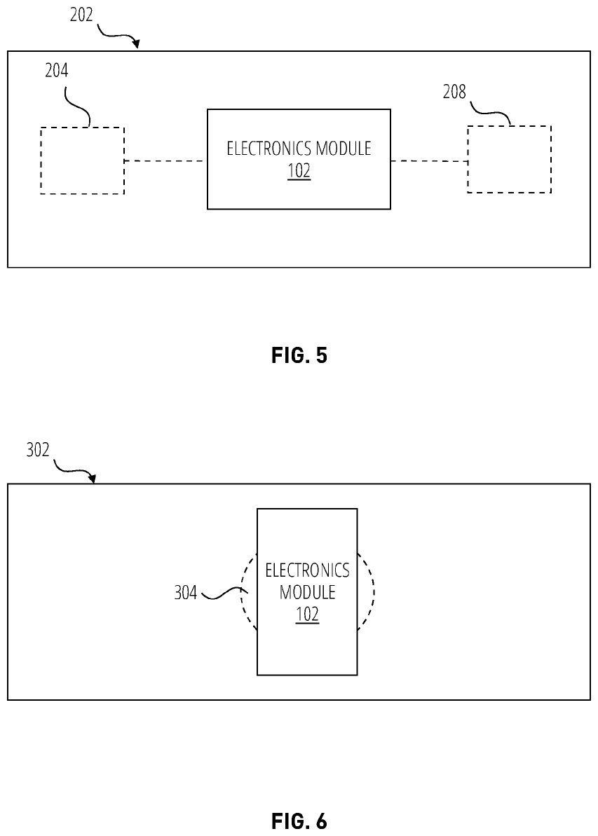

FIG. 5 shows the electronics module 102 coupled to the wearable article 202 of FIG. 2. In this example, the wearable article 202 comprises an electronics module holder such as a pocket (not shown) that holds the electronics module 102 in a first orientation. The controller 108 identifies from motion data recorded by the motion sensor that the electronics module 102 is in the first orientation. The controller 108 compares the first orientation to a look-up table stored in the memory of the electronics module 102 to identify that the electronics module 102 is coupled to a wearable article that comprises electrodes but no opening. The controller 108 configures the electronics module 102 to perform measurements using the electrical sensor 104 and not perform measurements using the optical sensor 106.

FIG. 6 shows the electronics module 102 coupled to the wearable article 302 of FIG. 3. In this example, the wearable article 302 comprises an electronics module holder such as a pocket (not shown) that holds the electronics module 102 in a second orientation different to the first orientation. In this example, the second orientation is perpendicular to the first orientation. This is of course not required in all examples. The controller 108 identifies from motion data recorded by the motion sensor that the electronics module 102 is in the second orientation. The controller 108 compares the second orientation to a look-up table stored in the memory of the electronics module 102 to identify that the electronics module 102 is coupled to a wearable article that comprises no electrodes but an opening. The controller 108 configures the electronics module 102 to perform measurements using the optical sensor 106 and not perform measurements using the electrical sensor 104.

In some examples, the controller 108 identifies from motion of the electronics module 102 where on the wearer the electronics module 102 is located. The controller 108 may identify, from the motion data, characteristic movements associated with different parts of the body such as the wrist, arm, leg or chest. The controller 108 may identify from these characteristic movements the location of the electronics module 102 on the wearer and the thus the type of wearable article the electronics module 102 is located on such as a wrist band, arm band/sleeve, leg sleeve, or top/chest band. The controller 108 may use a look-up table to determine how to operate the electrical sensor 104 and the optical sensor 106 based on this identification of the type of wearable article. For example, when the controller 108 identifies that the electronics module 102 is coupled to a wrist band, arm band/sleeve, or leg sleeve the controller 108 may perform measurements using the optical sensor 106 and not perform measurements using the electrical sensor 104. Meanwhile, when the controller 108 identifies that the electronics module 102 is coupled to a top/chest-band, the controller 108 may perform measurements using the electrical sensor 104 and not the optical sensor 106 or both the electrical sensor 104 and the optical sensor 106.

In some examples, the contextual information comprises an electrical measurement performed by the electrical sensor 104. The electrical measurement may be a voltage or impedance measurement for example.

Different electrical properties may be measured depending on the type of wearable article that the electronics module 102 is coupled to or the location of the electronics module 102 on the wearable article. The value recorded by the electrical sensor 104 may be compared to a look-up table to identify the type of wearable article that the electronics module 102 is coupled to or the location of the electronics module 102 on the wearable article. In other words, the value recorded by the electrical sensor 104 indicates whether the electrical sensor is connected to an electrode of the wearable article and whether the optical sensor has line of sight with a skin surface of the user.

The electrical measurement may have a first value when the electronics module 102 is coupled to a wearable article with electrodes such as wearable article 202 of FIG. 2. In this example, the first contact 110 and second contact 112 of the electrical sensor 104 are coupled to the first electrode 204 and the second electrode 208 and are able perform electrical measurements from the skin surface of the wearer via the first electrode 204 and the second electrode 208. The electrical measurements have a first value that is characteristic of the electrical sensor 104 being coupled to the first electrode 204 and the second electrode 208. It will be appreciated that the first value may be a range of values. Accordingly, the controller 108 controls the electronics module 102 to perform measurements using the electrical sensor 104 and not the optical sensor 106. The controller 108 may enable the use of a first signal processing algorithm for processing signals measured by the electrical sensor 104.

The electrical measurement may have a second value when the electronics module 102 is coupled to a wearable article without electrodes such as wearable article 302 of FIG. 3. In this example, the first contact 110 and the second contact 112 of the electrical sensor 104 are not coupled to electrodes of the wearable article 302 and are not in contact or close proximity with the skin of the wearer. Instead, the first contact 110 and second contact 112 are coupled to the fabric of the wearable article 302. The electrical measurements have a second value, different to the first value, that is characteristic of the electrical sensor 104 not being coupled to the electrodes or in contact or close proximity with the skin of the wearer. It will be appreciated that the second value may be a range of values. According, the controller 108 controls the electronics module 102 to perform measurements using the optical sensor 106 and not the electrical sensor 104.

The electrical measurement may have a third value when the electronics module is coupled to a wearable article without electrodes but which is arranged to allow the first contact 110 and the second contact 112 to contact or be in close proximity with the skin of the wearer such as the wearable article 402 of FIG. 4. The electrical measurement has a third value, different to the first value and the second value, that is characteristic of the first contact 110 and the second contact 112 being in contact or in close proximity with the skin of the wearer. Accordingly, the controller 108 controls the electronics module 102 to perform measurements using the electrical sensor 104 and the optical sensor 106. The controller 108 may enable the use of a second signal processing algorithm to process signals measured by the electrical sensor 104.

In some examples, a signal quality metric for the electrical measurement is used to determine the type of wearable article that the electronics module 102 is coupled to or location of the electronics module 102 on the wearable article. In other words, the signal quality metric indicates whether the electrical sensor is connected to an electrode of the wearable article and whether the optical sensor has line of sight with a skin surface of the user. If the first contact 110 and second contact 112 of the electrical sensor 104 are coupled to electrodes of the wearable article, then the signal quality is expected to be high and above a threshold value. If the first contact 110 and second contact 112 of the electrical sensor 104 are not coupled to electrodes of the wearable article or in close contact or proximity with the skin, then the signal quality is expected to be low and below a threshold value. Accordingly, the controller 108 controls the electronics module 102 to perform measurements using the electrical sensor 104 when the signal quality is above the threshold value and controls the electronics module 102 to perform measurements using the optical sensor 106 when the signal quality is below the threshold value.

In some examples, the signal quality of the electrical measurement may be greater than a first threshold value when the first contact 110 and the second contact 112 are coupled to electrodes of the wearable article (as per FIG. 2). The signal quality of the electrical measurement may be less than the first threshold value and greater than a second threshold value when the first contact 110 and the second contact 112 are not coupled to electrodes of the wearable article but are in contact with or close proximity with the skin of the wearer (as per FIG. 4). The signal quality of the electrical measurement may be less than the second threshold value when the first contact 110 and the second contact 112 are not coupled to electrodes of the wearable article and are not in contact with or close proximity with the skin of the wearer (as per FIG. 3).

Accordingly, the controller 108 controls the electronics module 102 to perform measurements using the electrical sensor 104 and not the optical sensor 106 when the signal quality of the electrical measurement is greater than the first threshold value, controls the electronics module 102 to perform measurements using the electrical sensor 104 and the optical sensor 106 when the signal quality of the electrical measurement is less than the first threshold value and greater than the second threshold value, and controls the electronics module 102 to perform measurements using the optical sensor 106 and not the electrical sensor 104 when the signal quality of the electrical measurement is less than the second threshold value.

In some examples, the contextual information comprises capacitance data measured by a capacitive sensor of the electronics module 102. The capacitive sensor is arranged to measure a change in capacitance value based on the capacitive properties of materials in the vicinity of the electronics module 102.

Different materials have different capacitance properties. Different types of wearable articles can be constructed or otherwise incorporate, at least in the vicinity of the electronics module holder that retains the electronics module 102, from different types of materials. A single wearable article may use different materials at different locations on the wearable article. The capacitance sensor can measure the capacitance value to determine the type of wearable article that the electronics module 102 is coupled to or the location of the electronics module 102 on a wearable article.

When coupled to the wearable article 202 of FIG. 2, the capacitance sensor may measure a first capacitance value. The first capacitance value may be due to the presence of the conductive first connection region 206 and the conductive second connection region 210. The first capacitance value may also be due to the material properties of the wearable article 202 such as the type of fabric used and the presence of other material such as silicone, polyvinyl chloride, synthetic or natural rubber, epoxy resin or polyamide. The first capacitance value may be a range of capacitance values.

When coupled to the wearable article 302 of FIG. 3, the capacitance sensor may measure a second capacitance value different to the first capacitance value. The second capacitance value may be due to the absence of conductive connection regions. The second capacitance value may also be due to the material properties of the wearable article 302. The second capacitance value may be a range of capacitance values.

When coupled to the wearable article 402 of FIG. 4, the capacitance sensor may measure a third capacitance value different to the first and second capacitance value. The third capacitance value may be due to the first contact 110 and the second contact 112 of the electronics module 102 contacting or coming in to close proximity with the skin surface. The third capacitance value may also be due to the material properties of the wearable article 402. The third capacitance value may be a range of capacitance values.

Accordingly, the controller 108 obtains the capacitance value from the capacitive sensor and compares the capacitance value to a look-up table to determine the type of wearable article that the electronics module 102 is coupled to or the location of the electronics module 102 on a wearable article. In other words, the capacitance value indicates whether the electrical sensor is connected to an electrode of the wearable article and whether the optical sensor has line of sight with a skin surface of the user. The controller 108 controls the electronics module 102 to perform measurements using the electrical sensor 104 and not the optical sensor 106 when the capacitance sensor measures the first capacitance value. The controller 108 controls the electronics module 102 to perform measurements using the optical sensor 106 and not the electrical sensor 104 when the capacitance sensor measures the second capacitance value. The controller 108 controls the electronics module 102 to perform measurements using the electrical sensor 104 and the optical sensor 106 when the capacitance sensor measures the third capacitance value.

In some examples, the contextual information comprises electrostatic data measured by an electrostatic sensor of the electronics module 102. An example electrostatic sensor is the Qvar electrostatic sensor manufactured by STMicroelectronics.

The electrostatic sensor can be used in a similar way to the capacitance sensor described above to identify different material properties in the vicinity of the electronics modules 102. Different types of wearable articles can be constructed or otherwise incorporate, at least in the vicinity of the electronics module holder that retains the electronics module 102, from different types of materials. A single wearable article may use different materials at different locations on the wearable article. The electrostatic sensor can measure the electrostatic value to determine the type of wearable article that the electronics module 102 is coupled to or the location of the electronics module 102 on the wearable article. In other words, the electrostatic value indicates whether the electrical sensor is connected to an electrode of the wearable article and whether the optical sensor has line of sight with a skin surface of the user. The controller obtains the electrostatic value from the electrostatic sensor and compares the capacitance value to a look-up table to determine the type of the wearable article that the electronics module 102 is coupled to or the location of the electronics module 102 on the wearable article. The controller 108 controls the electronics module 102 to perform measurements using the electrical sensor 104 and/or optical sensor 106 according to the determined type of wearable article.

In some examples, the contextual information comprises force data indicative of a force applied to the electronics module. The force data may be measured by a force sensor of the electronics module 102.

Different types of wearable article may compress the electronics module 102 by different amounts. The electronics module 102 may be compressed by different amounts at different locations on the wearable article. The amount of compression applied to the electronics module 102 is measured by the force sensor. The controller 108 obtains the force data and compares the force data to a look-up table to determine the type of wearable article that electronics module 102 is coupled to or the location of the electronics module 102 on the wearable article. In other words, the force data indicates whether the electrical sensor is connected to an electrode of the wearable article and whether the optical sensor has line of sight with a skin surface of the user. The controller 108 controls the electronics module 102 to perform measurements using the electrical sensor 104 and/or optical sensor 106 according to the determined type of wearable article.

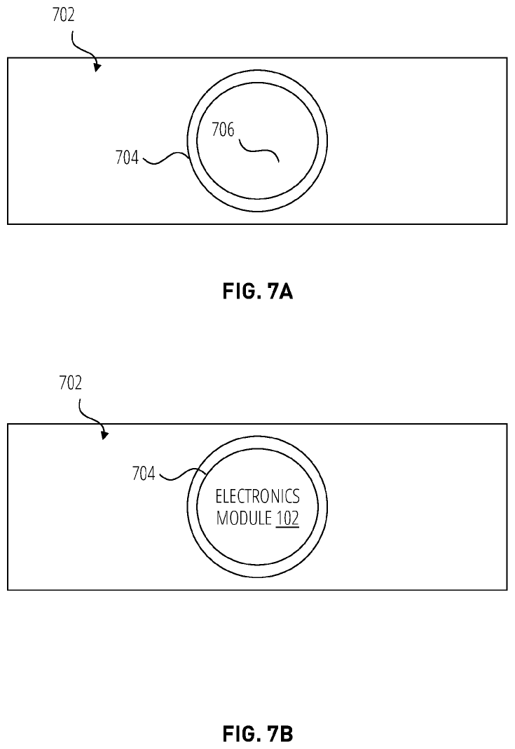

FIG. 7A shows an example wearable article 702 for receiving the electronics module 102. The wearable article 702 may be in the form of a wrist strap. The wearable article 702 comprises a frame 704 which bounds an opening 706. The frame 704 may be a plastic injection moulded frame.

The frame 704 receives the electronics module 102 and forms a snap-fit engagement with the electronics module 102 to hold the electronics module 102 in place. As the electronics module 102 is received within the opening 706 of the wearable article 702 the optical sensor 106 of the electronics module 102 has line of sight with the skin surface of the wearer. The wearable article 702 in this example does not comprise electrodes. The frame 704 and the electronics module 102 comprise complementary interlocking grooves and protrusions for retaining the electronics module 102 within the frame 704. The frame 704 may also comprise a release mechanism for releasing the electronics module 102 from the frame 704.

FIG. 7B shows the electronics module 102 positioned within the frame 704. The frame 704 applies compression to the electronics module 102 which may cause part of the housing of the electronics module 102 to deform. This deformation can be detected by a force sensor of the electronics module 102.

When coupled to another type of wearable article such as the wearable article 202 of FIG. 2, the electronics module 102 may not be under compression or the compression may be of a different amount or from a different direction. The compressive force could be applied to the top of the housing rather than the sides of the housing as shown in FIG. 7A and FIG. 7B. The force sensor is able to detect a different amount of compression being applied when coupled to different wearable articles to determine the type of wearable article that the electronics module 102 is coupled to. The controller 108 is able to control the operation of the electrical sensor 104 and the optical sensor 106 accordingly. For example, when the force sensor detects compression being applied by the frame 704, the controller 108 can perform measurements using the optical sensor 106 and not the electrical sensor 104. Meanwhile, when the force sensor does not detect compression being applied to the electronics module 102, the controller 108 can perform measurements using the electrical sensor 104 and not the optical sensor 106.

The force sensor may be any form of force sensor as known in the art. The force sensor may be an inductive force sensor. An inductive force sensor may comprise a metal target and a coil which are separated from one another. When the housing is compressed, the distance between the metal target and the coil decreases and the change in electrical properties are measured by the force sensor. An example force sensor is the Azoteq Force Touch by Azoteq (Pyt) Ltd.

In some examples, the contextual information comprises an optical measurement performed by the optical sensor 106 or another optical sensor of the electronics module 102.

For some types of wearable article, such as the wearable article 202 of FIG. 2, the optical sensor 106 is separated from the skin surface by the wearable article 202 and does not have line of sight with the skin surface. Other wearable articles, such as the wearable article 302 of FIG. 3, the wearable article 402 of FIG. 4, and the wearable article 702 of FIG. 7A have an opening 304, 404, 706 that allows the optical sensor 106 to have line of sight with the skin surface. The optical measurement performed by the optical sensor 106 will have a different value dependent on whether the optical sensor 106 has line of sight with the skin surface.

When coupled to the wearable article 202 of FIG. 2, the optical measurement may have a first value. The first value may be a range of values. The first value may indicate that the optical sensor 106 does not have line of sight with the skin surface. The fabric of the wearable article 202 in the vicinity of the optical sensor 106 may have a particular colour or a particular property (e.g., being highly reflective) to help enable the electronics module 102 to distinguish from measurements of the skin surface. Different wearable articles may have different colours or other properties (e.g., degrees of reflectivity) to allow for the electronics module 102 to identify different types of wearable article that the electronics module 102 is coupled to. Likewise, different locations on a single wearable article may have different colours or other properties to allow for the location of the electronics module 102 on the wearable article to be identified.

When coupled to the wearable article 302 of FIG. 3, the wearable article 402 of FIG. 4, and the wearable article 702 of FIG. 7A, the optical measurement may have a second value. The second value may be a range of values. The second value may indicate that the optical sensor 106 does have line of sight with the skin surface.

Likewise, a single wearable article may retain an electronics module 102 at different locations. At one or more of the locations, the optical sensor has line of sight with the skin surface. At one or more of the locations, the optical sensor is obscured by the wearable article and does not have line of sight with the skin surface.

Accordingly, the controller 108 obtains the optical measurement from the optical sensor 106 and compares the value to a look-up table to determine the type of wearable article that the electronics module 102 is coupled to or the location of the electronics module on the wearable article. In other words, the optical measurement indicates whether the electrical sensor is connected to an electrode of the wearable article and whether the optical sensor has line of sight with a skin surface of the user. The controller 108 controls the electronics module 102 to perform measurements using the electrical sensor 104 and not the optical sensor 106 when the optical measurement has the first value. The controller 108 controls the electronics module 102 to perform measurements using the optical sensor 106 and not the electrical sensor 104 when the optical measurement has the second value.

In some examples, the contextual information comprises magnetic field data measured by a magnetic field sensor of the electronics module 102. Some types of wearable article or locations on a single wearable article may incorporate a magnetic material such as a permanent magnetic. When coupled to these types of wearable article or locations, the magnetic field sensor detects a magnetic field. The controller 108 is able to identify the type of wearable article or location of the electronics module on the wearable article based on whether a magnetic field is detected and configured to control the operation of the electrical sensor 104 and the optical sensor 106 accordingly. The magnetic field sensor may be a half-effect sensor, magnetometer or reed sensor for example.

In some examples, the wearable article may comprise a location tag such as an NFC tag that contains information indicative of the type of wearable article or location on the wearable article. The electronics module 102 may comprise a reader (e.g. an NFC reader) arranged to read the location tag. The tag may be located in the vicinity of the electronics module holder such that the electronics module 102 reads the tag when coupled to the electronics module holder.

In some examples, the electronics module 102 may comprise one or more switches protruding from the housing of the electronics module 102. Different wearable articles comprise different features for engaging with the switches. In one example, the electronics module 102 comprises a single switch. Wearable articles suitable for optical measurements comprise a feature that engages with the switch when the electronics module is coupled to the wearable article. The electronics module 102 determines that the switch is closed and configures the operation of the electronics module 102 accordingly. Wearable articles not suitable for optical measurements do not comprise features for engaging with the switch, The electronics module 102 determines that the switch is open and configures the operation of the electronics module 102 accordingly. It will be appreciated that the electronics module 102 may comprise a plurality of switches and different wearable articles may comprise different features or groups of features to engage with different combinations of switches. This allows for identification of a greater variety of types of wearable articles.

In some examples, the wearable article may comprise a non-volatile memory which is read by the electronics module 102 when the electronics module 102 is coupled to the wearable article. The non-volatile memory may store information identifying the type of wearable article. Example implementations of incorporating non-volatile memories in wearable articles are described in International Patent Application Publication No. WO2021/105684.

Any combination of the different types of contextual information described above may be used to determine the type of wearable article that the electronics module 102 is coupled to or the location of the electronics module on the wearable article. In other words, any combination of the different types of contextual information described above may be used to determine whether the electrical sensor is connected to an electrode of the wearable article and whether the optical sensor has line of sight with a skin surface of the user.

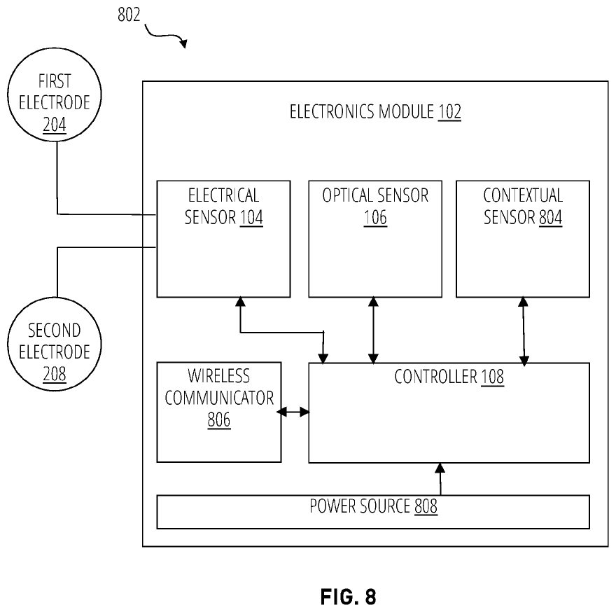

FIG. 8 shows a schematic diagram for an example electronics module 102 according to aspects of the present disclosure.

The electronics module 102 comprises an electrical sensor 104, an optical sensor 106, and a controller 108 as described above.

The electrical sensor 104 is shown in communicative coupling with a first electrode 204 and a second electrode 208 of a wearable article. The electrodes 204, 208 are placed in contact with a skin surface of a user. The electrical sensor 104 receives analogue signals from the electrodes 204, 208 and converts the analogue signals into digital signal values. The electrical sensor 104 may also perform additional processing on the signals such as for noise reduction.

The controller 108 comprises a processor and a memory. The controller 108 controls the operation of the electronics module 102. The controller 108 may comprises a plurality of processors. The controller 108 may comprise an application processor and a machine learning processor (e.g., a machine learning core). The components of the controller 108 may be distributed in the electronics module 102 and are not required to be provided in a single integrated circuit package.

The electronics module 102 further comprises a contextual sensor 804. The contextual sensor 804 may be any of or a combination of the contextual sensors described above.

The electronics module further comprises a wireless communicator 806 and a power source 808 for supplying power to the components of the electronics module 102.

The wireless communicator 806 is arranged to communicatively couple with an external device over one or more wireless communication protocols. The wireless communication protocol may be a Bluetooth® protocol, Bluetooth® 5 or a Bluetooth® Low Energy protocol but is not limited to any particular communication protocol. The wireless communicator 806 enables communication between the external device and the controller 108 for configuration and set up of the controller 108 and the peripheral devices as may be required. Configuration of the controller 108 and peripheral devices utilises the Bluetooth® protocol in this example.

Other wireless communication protocols can also be used, such as used for communication over: a wireless wide area network (WWAN), a wireless metro area network (WMAN), a wireless local area network (WLAN), a wireless personal area network (WPAN), Bluetooth® Low Energy, Bluetooth® Mesh, Thread, Zigbee, IEEE 802.15.4, Ant, a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network.

The power source 808 may comprise one or a plurality of power sources. The power source 808 may be a battery. The battery may be a rechargeable battery. The battery may be a rechargeable battery adapted to be charged wirelessly such as by inductive charging. The power source 808 may comprise an energy harvesting device. The energy harvesting device may be configured to generate electric power signals in response to kinetic events such as kinetic events performed by the wearer of the wearable article. The kinetic event could include walking, running, exercising or respiration of the wearer. The energy harvesting material may comprise a piezoelectric material which generates electricity in response to mechanical deformation of the converter. The energy harvesting device may harvest energy from body heat of the wearer. The energy harvesting device may be a thermoelectric energy harvesting device. The power source 808 may be a super capacitor, or an energy cell.

The electronics module 102 may additionally comprise a power receiving interface operable to receive power from an external power store for charging the power source 808. The power receiving interface may be a wired or wireless interface. A wireless interface may comprise one or more wireless power receiving coils for receiving power from the external power store.

The power receiving interface may also be coupled to the controller 108 to enable direct communication between the controller 108 and an external device if required.

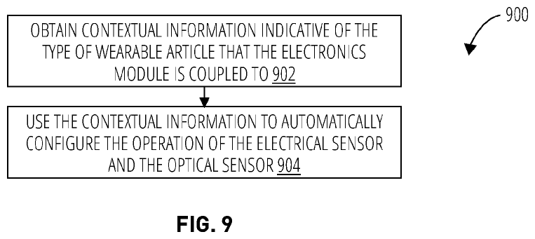

FIG. 9 shows an example method according to aspects of the present disclosure. In step 902, method 900 obtains contextual information indicative of the type of wearable article that the electronics module 102 is coupled to or location of the electronics module 102 on the wearable article. In step 904, method 900 uses the contextual information to automatically configure the operation of the electrical sensor 104 and the optical sensor 106.

The method may be performed each time the electronics module 102 is coupled to a different wearable article or is coupled to a different location on a single wearable article.

In an example operation, the electronics module 102 is coupled a first type of wearable article. The method 900 obtains contextual information indicative of the type of wearable article that the electronics module 102 is coupled to and uses the contextual information to automatically configure the operation of the electrical sensor 104 and the optical sensor 106.

Subsequently, the electronics module 102 is coupled to a second type of wearable article. The method 900 obtains contextual information indicative of the type of wearable article that the electronics module 102 is coupled to and uses the contextual information to automatically configure the operation of the electrical sensor 104 and the optical sensor 106.

The above examples generally refer to configuring the operation of the electrical sensor 104 and optical sensor 106 depending on the type of wearable article that the electronics module 102 is coupled to. In some examples, the electronics module 102 may configure its operation depending on its location on a wearable article. For example, at a first location on the wearable article, the electronics module 102 may perform monitoring using the optical sensor 106 and not the electrical sensor 104 while at a second location on the wearable article, the electronics module 102 may perform monitoring using the electrical sensor 104 and not the optical sensor 106.

FIG. 10A and FIG. 10B show an example wearable article 1002 in the form of a t-shirt.

The wearable article 1002 comprises a first electronics module holder 1004 and a second electronics module holder 1006 at different locations on the wearable article 1002. The first electronics module holder 1004 is located on the centre-front of the t-shirt and the second electronics module holder 1006 is located on a sleeve of the t-shirt. The first electronics module holder 1004 and second electronics module holder 1006 are pockets in this example. The second electronics module holder 1006 holds the electronics module 102 in a different orientation to the first electronics module holder 1004. The electronics module 102 is held horizontally in the first electronics module holder 1004 and vertically in the second electronics module holder 1006.

The wearable article 1002 comprises a first electrode 1008, first conductive pathway 1010, and first connection region 1012. The wearable article 1002 comprises a second electrode 1014, second conductive pathways 1016, and second connection region 1018. The first connection region 1012 and second connection region 1018 are accessible by the electronics module 102 when coupled to the first electronics module holder 1004 (positioned in the pocket 1004) in this example.

The wearable article 1002 comprises an opening 1020 for the optical sensor 106 of the electronics module 102. When the electronics module 102 is coupled to the second electronics module holder 1006, the optical sensor 106 has line of sight through the opening 1020.

The electronics module 102 may determine from contextual information, as described above, whether it is coupled to the first electronics module holder 1004 or the second electronics module holder 1006 and may configure its operations accordingly.

For example, when coupled to the first electronics module holder 1004, the electronics module 102 performs measurements using the electrical sensor 104 and not the optical sensor 106 and when coupled to the second electronics module holder 1006, the electronics module 102 performs measurements using the optical sensor 106 and not the electrical sensor 104. The electronics module 102 may also perform measurements using the electrical sensor 104 when coupled to the second electronics module holder 1006 if the opening 1020 is sized to enable the first contact 110 and second contact 112 to contact with or be positioned in close proximity to the skin surface as described above in relation to FIG. 4. The electronics module 102 may additionally or separately perform measurements using a hidden temperature sensor as explained below in relation to FIG. 12, FIG. 13 and FIG. 14.

FIG. 11A and FIG. 11B show another example wearable article 1102 in the form of a bra 1102.

The wearable article 1102 comprises a first electronics module holder 1104 and a second electronics module holder 1106 at different locations on the wearable article 1102. The first electronics module holder 1104 is located on the centre-front of the underband 1108 of the bra 1102 and the second electronics module holder 1106 is located in a side region of the bra 1102. The first electronics module holder 1104 and second electronics module holder 1106 are pockets in this example. The second electronics module holder 1106 holds the electronics module 102 in a different orientation to the first electronics module holder 1104. The electronics module 102 is held horizontally in the first electronics module holder 1104 and vertically in the second electronics module holder 1106.

The wearable article 1102 comprises a first electrode 1110, first conductive pathway 1112, and first connection region 1114 located on the underband 1108. The wearable article 1102 comprises a second electrode 1116, second conductive pathway 1118, and second connection region 1120 located on the underband 1108. The first connection region 1114 and second connection region 1120 are accessible by the electronics module 102 when coupled to the first electronics module holder 1104 (positioned in the pocket 1104) in this example.

The wearable article 1102 comprises an opening 1122 for the optical sensor 106 of the electronics module 102. When the electronics module 102 is coupled to the second electronics module holder 1106, the optical sensor 106 has line of sight through the opening 1020.

The electronics module 102 may determine from contextual information, as described above, whether it is coupled to the first electronics module holder 1104 or the second electronics module holder 1106 and may configure its operations accordingly.

For example, when coupled to the first electronics module holder 1004, the electronics module 102 performs measurements using the electrical sensor 104 and not the optical sensor 106 and when coupled to the second electronics module holder 1006, the electronics module 102 performs measurements using the optical sensor 106 and not the electrical sensor 104. The electronics module 102 may also perform measurements using the electrical sensor 104 when coupled to the second electronics module holder 1006 if the opening 1020 is sized to enable the first contact 110 and second contact 112 to contact with or be positioned in close proximity to the skin surface as described above in relation to FIG. 4. The electronics module 102 may additionally or separately perform measurements using a hidden temperature sensor as explained below in relation to FIG. 12, FIG. 13 and FIG. 14.

In another example bra construction, the first electronics module holder 1104 and second electronics module holder 1106 may both be located on the underband 1108.

It will be appreciated that other wearable article constructions besides t-shirts and bras are possible. Other types of wearable article and in particular other forms of clothing may include the same multiple electronics module holder construction. The location of the first and second electronics module holder will depend on the construction of the particular wearable article.

Generally, the second electronics module holder, which is used by the electronics module 102 to perform optical measurements, will be located at a position suitable for performing optical measurements of the wearer. These locations are generally where the pulse rate can be clearly measured.

One example measurement location is the upper arm such as in the vicinity of the brachial artery. The wearable article may be a top that comprises at least one sleeve. The second electronics module holder is located on the sleeve such that the optical sensor 106 of the electronics module 102 can be positioned in the vicinity of the brachial artery.

Another example measurement location is the left side of the torso. The wearable article may be a top such as a shirt or bra and the second electronics module holder may be located on a left side region of the top.

Another example measurement location is the lower leg in the vicinity of the popliteal artery. The wearable article may be a pair of trousers, tights or leggings. The second electronics module holder is located such that the optical sensor 106 of the electronics module 102 can be positioned in the vicinity of the popliteal artery.

Another example measurement location is the waist in the vicinity of the femoral artery. The wearable article may be a pair of trousers, tights, leggings, shorts or underwear. The second electronics module holder may be located on the wearable article such that the optical sensor 106 of the electronics module 102 can be positioned in the vicinity of the femoral artery. Generally, this means that the second electronics module holder is located in a waistband of the wearable article.

It will be appreciated that a wearable article may comprise multiple of the second electronics module holders located at different positions on the wearable article to enable optical measurements to be performed at multiple locations. The measurements may be performed simultaneously if a plurality of electronics modules are simultaneously retained in the plurality of electronics module holders or successively if an electronics module is swapped between different ones of the electronics module holders.

FIG. 12 shows the bottom surface of an example electronics module 102 according to aspects of the present disclosure. The optical sensor 106 is centrally located between the first contact 110 and second contact 112. The first contact 110 and second contact 112 are in the form of contact pads which may be flat or textured. The contact pads may project from the bottom surface of the electronics module 102 or may be substantially flush with the bottom surface of the electronics module 102. Charging pins 1202, 1204 for charging the electronics module 102 are located around the optical sensor 106.

FIG. 13 shows the electronics module 102 in FIG. 12 with one of the contact pads 110 removed.

The first conductor 114 extends through the bottom surface of the housing of the electronics module 102 to electrically couple the first contact 110 to the processor within the housing. The first conductor 114 is in the form of a spring-loaded pin also known as a pogo pin. In a preferred example, the pogo pins are suitable to be applied using a surface mount technology which lowers manufacturing costs. An example of such as pogo pin is the P70-2000045R pogo pin from Harwin PLC. Such surface mount suitable pogo pins may include additional locating pins for use in the surface mount process. These locating pins may, beneficially, provide additional structural support and reduce translational movement of the pogo pins relative to the printed circuit board.

In addition, a temperature sensor 1302 is sandwiched between the housing and the first contact 110. This arrangement of the temperature sensor 1302 is described in greater detail in UK Patent No. GB2592694 in the name of Prevayl Innovations Limited, the disclosures of which are herein incorporated by reference.

The temperature sensor 1302 is arranged to monitor a skin-surface temperature of the user wearing the electronics module 102. The temperature sensor 1302 is a contact temperature sensor such as a thermocouple or thermistor. The temperature sensor 1302 does not directly contact the skin and instead is in thermal contact with the first contact 110. The first contact 110 is thermally conductive and acts a coupling medium to couple the temperature sensor 1302 to the point of measurement (e.g., the skin-surface). Example thermally conductive materials usable for the first contact 110 include aluminium and copper. Of course, other electrically and thermally conductive materials may be used. The first contact 110 therefore covers the temperature sensor 1302 and first conductor 114 and protects these components against damage. The first contact 110 covers the first conductor 114 and temperature sensor 1302 and seals of the housing of the electronics module 102 to help protect against water ingress into the housing.

A temperature sensor may also be provided underneath the second contact 112. That is a second temperature sensor may be sandwiched between the housing of the electronics module 102 and the second contact 112.

FIG. 14 shows an example wearable article 1402 according to aspects of the present disclosure. The wearable article 1402 comprises an electronics module holder 1404 in the form of a pocket 1404. The pocket 1404 has an open end 1408 that may be selectively closed using a fastener such as Velcro. The pocket 1404 also has an opening 1406 which exposes part of the electronics module 102 when positioned in the pocket 1404. The opening 1406 is bounded by a strip of reinforcement material 1410.

As shown in FIG. 14, the electronics module 102 is positioned in the pocket 1404 such that the bottom surface of the electronics module 102 faces the opening 1406 and is partially exposed by the opening 1406. Components of the electronics module 102 or parts thereof are exposed may extend through the opening 1406 such as the optical sensor 106 and first contact 110 and second contact 112 to contact or be in close proximity to the skin to enable measurements to be performed.

As explained above in relation to FIG. 4, the first and second contacts 110, 112 extend through the opening 1406 to contact the skin to enable electrical measurements to be performed by the electrical sensor 104. In this example, the first and second contacts 110, 112 are only partially exposed by the opening 1406, but the surface area of exposure can still be sufficient to enable measurements of the skin surface to be made.

Alternatively, or separately, thermal measurements can be made using one or more temperature sensors 1302 of the electronics module 102 which are covered by either or both of the first contact 110 and the second contact 112. The first contact 110 and second contact 112 are partially exposed and able to contact the skin to thermally couple the temperature sensor 1302 to the skin surface. This enables a skin surface temperature measurement to be performed.

It will be appreciated that if a temperature sensor 1302 is only provided behind one of the contacts 110, 112, then the contact 110, 112 covering the temperature sensor 1302 need be exposed by the opening 1406 to contact the skin. However, exposing all or part of both contacts 110, 112 is generally preferred as it allows for both temperature and electrical measurements to be performed.

In summary, there is provided an electronics module 102 and method. The electronics module 102 is for monitoring physical activity of a user. The electronics module 102 comprises an electrical sensor 104, an optical sensor 106 and a controller 108. The controller 108 obtains contextual information indicative of the type of wearable article that the electronics module 102 is coupled to or the location of the electronics module on the wearable article. The controller 108 is arranged to use the contextual information to automatically configure the operation of the electrical sensor 104 and the optical sensor 106. In some examples, the controller 108 automatically switches between performing measurements using the electrical sensor 104 and the optical sensor 106 based on the contextual information. The contextual information may indicate whether the electrical sensor 104 is coupled to one or more electrodes of a wearable article.

### EXAMPLES

Example 1. An electronics module for monitoring physical activity of a user, the electronics module comprising an electrical sensor and an optical sensor, and a controller arranged to obtain contextual information indicative of the type of wearable article the electronics module is coupled to, and use the contextual information to automatically configure the operation of the electrical sensor and the optical sensor.

Example 2. The electronics module of Example 1, wherein the controller is arranged to use the contextual information to automatically switch the electronics module between performing measurements using the electrical sensor and the optical sensor.

Example 3. The electronics module of Example 2, wherein the controller is arranged to automatically switch the electronics module between a first measurement mode, in which the electrical sensor is used to perform measurements and the optical sensor is not used to perform measurements, and a second measurement mode, in which the electrical sensor is not used to perform measurements and the optical sensor is used to perform measurements.

Example 4. The electronics module of any one of Examples 1 to 3, wherein the contextual information is indicative of whether the electrical sensor is coupled to one or more electrodes of a wearable article.

Example 5. The electronics module of Example 4, wherein the controller is arranged to perform measurements using the electrical sensor when the contextual information indicates that the electrical sensor is coupled to one or more electrodes of a wearable article, and wherein the controller is arranged to perform measurements using the optical sensor when the contextual information indicates that the electrical sensor is not coupled to one or more electrodes of a wearable article.

Example 6. The electronics module of any one of Examples 1 to 5, wherein the contextual information comprises motion data for the electronics module.

Example 7. The electronics module of Example 6, wherein the motion data identifies the orientation of the electronics module.

Example 8. The electronics module of Example 7, wherein the motion data indicates that the electronics module is coupled to a first type of wearable article when the motion data identifies the electronics module as being in a first orientation, and wherein the motion data indicates that electronics module is coupled to a second type of wearable article when the motion data identifies the electronics module as being in a second orientation different to the first orientation.

Example 9. The electronics module of any one of Examples 1 to 7, wherein the contextual information comprises an electrical measurement performed by the electrical sensor.

Example 10. The electronics module of Example 9, wherein the electrical measurement indicates that the electronics module is coupled to a first type of wearable article when the electrical measurement has a first value, and wherein the electrical measurement indicates that the electronics module is coupled to a second type of wearable article when the electrical measurement has a second value different to the first value.

Example 11. The electronics module of Example 9 or 10, wherein the electrical measurement indicates that the electronics module is coupled to a first type of wearable article when the electrical measurement has a signal quality above a threshold value, and wherein the electrical measurement indicates that the electronics module is coupled to a second type of wearable article when the electrical measurement has a signal quality below the threshold value.

Example 12. The electronics module of any one of Examples 1 to 11, wherein the contextual information comprises capacitance data measured by a capacitive sensor of the electronics module.

Example 13. The electronics module of Example 12, wherein the capacitance data indicates that the electronics module is coupled to a first type of wearable article when the capacitance data has a first value, and wherein the capacitance data indicates that the electronics module is coupled to a second type of wearable article when the capacitance data has a second value different to the first value.

Example 14. The electronics module of any one of Examples 1 to 13, wherein the contextual information comprises electrostatic data measured by an electrostatic sensor of the electronics module.

Example 15. The electronics module of Example 14, wherein the electrostatic data indicates that the electronics module is coupled to a first type of wearable article when the electrostatic data has a first value, and wherein the electrostatic data indicates that the electronics module is coupled to a second type of wearable article when the electrostatic data has a second value different to the first value.

Example 16. The electronics module of any one of Examples 1 to 15, wherein the contextual information comprises force data indicative of a force applied to the electronics module.

Example 17. The electronics module of Example 16, wherein the force data indicates that the electronics module is coupled to a first type of wearable article when the force data has a first value, and wherein the force data indicates that the electronics module is coupled to a second type of wearable article when the force data has a second value different to the first value.

Example 18. The electronics module of any one of Examples 1 to 17, wherein the contextual information comprises an optical measurement performed by the optical sensor.

Example 19. The electronics module of Example 18, wherein the optical measurement indicates that the electronics module is coupled to a first type of wearable article when the optical measurement has a first value, and wherein the optical measurement indicates that the electronics module is coupled to a second type of wearable article when the optical measurement has a second value different to the first value.

Example 20. The electronics module of any one of Examples 1 to 19, wherein the contextual information comprises magnetic field data measured by a magnetic field sensor of the electronics module.

Example 21. The electronics module of Example 20, wherein the magnetic field data indicates that the electronics module is coupled to a first type of wearable article when the magnetic field data has a first value, and wherein the magnetic field data indicates that the electronics module is coupled to a second type of wearable article when the magnetic field data has a second value different to the first value.

Example 22. The electronics module of any one of Examples 1 to 21, wherein the electrical sensor comprises a bioelectrical and/or bioimpedance sensor.

Example 23. The electronics module of any one of Examples 1 to 22, wherein the optical sensor comprises a PPG sensor.

Example 24. A method performed by an electronics module for monitoring physical activity of a user, the method comprising: obtaining contextual information indicative of the type of wearable article that the electronics module is coupled to; and using the contextual information to automatically configure the operation of an electrical sensor and an optical sensor of the electronics module.

Example 25. A method performed by an electronics module for monitoring physical activity of a user, the method comprising: coupling the electronics module to a first type of wearable article; obtaining contextual information indicative of the type of wearable article that the electronics module is coupled to; using the contextual information to automatically configure the operation of an electrical sensor and an optical sensor of the electronics module; coupling the electronics module to a second type of wearable article; obtaining contextual information indicative of the type of wearable article that the electronics module is coupled to; and using the contextual information to automatically configure the operation of the electrical sensor and the optical sensor.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1-26. (canceled)

27. An electronics module for monitoring physical activity of a user, the electronics module being arranged to be removably coupled to a wearable article, the electronics module comprising:
an electrical sensor;
an optical sensor; and
a controller arranged to obtain contextual information from one or more sensors of the electronics module when the electronics module is coupled to the wearable article, and use the contextual information to automatically configure the operation of the electrical sensor and the optical sensor,
wherein the contextual information is indicative of whether the electrical sensor is connected to an electrode of the wearable article and whether the optical sensor has line of sight with a skin surface of the user,
wherein when the contextual information indicates that the electrical sensor is connected to an electrode of the wearable article, the controller is arranged to control the electrical sensor to perform measurements, and
wherein when the contextual information indicates that the optical sensor has line of sight with the skin surface of the user, the controller is arranged to control the optical sensor to perform measurements.

28. The electronics module of claim 27, wherein the controller is arranged to use the contextual information to automatically switch the electronics module between performing measurements using the electrical sensor and the optical sensor.

29. The electronics module of claim 28, wherein the controller is arranged to automatically switch the electronics module between a first measurement mode, in which the electrical sensor is used to perform measurements and the optical sensor is not used to perform measurements, and a second measurement mode, in which the electrical sensor is not used to perform measurements and the optical sensor is used to perform measurements.

30. The electronics module of claim 29, wherein the contextual information comprises motion data for the electronics module; wherein the motion data identifies the orientation of the electronics module.

31. The electronics module of claim 30, wherein the motion data indicates that the electrical sensor is connected to an electrode of the wearable article when the motion data identifies the electronics module as being in a first orientation, and wherein the motion data indicates that the optical sensor has line of sight with the skin surface of the user when the motion data identifies the electronics module as being in a second orientation different to the first orientation.

32. The electronics module of claim 30, wherein the contextual information comprises an electrical measurement performed by the electrical sensor.

33. The electronics module of claim 32, wherein the electrical measurement indicates that the electrical sensor is connected to an electrode of the wearable article when the electrical measurement has a first value, and wherein the electrical measurement indicates that the optical sensor has line of sight with the skin surface of the user when the electrical measurement has a second value different to the first value.

34. The electronics module of claim 33, wherein the electrical measurement indicates that the electrical sensor is connected to an electrode of the wearable article when the electrical measurement has a signal quality above a threshold value, and wherein the electrical measurement indicates that the optical sensor has line of sight with the skin surface of the user when the electrical measurement has a signal quality below the threshold value.

35. The electronics module of claim 34, wherein the contextual information comprises capacitance data measured by a capacitive sensor of the electronics module.

36. The electronics module of claim 35, wherein the capacitance data indicates that the electrical sensor is connected to an electrode of the wearable article when the capacitance data has a first value, and wherein the capacitance data indicates that the optical sensor has line of sight with the skin surface of the user when the capacitance data has a second value different to the first value.

37. The electronics module of claim 36, wherein the contextual information comprises electrostatic data measured by an electrostatic sensor of the electronics module.

38. The electronics module of claim 37, wherein the electrostatic data indicates that the electrical sensor is connected to an electrode of the wearable article when the electrostatic data has a first value, and wherein the electrostatic data indicates that the optical sensor has line of sight with the skin surface of the user when the electrostatic data has a second value different to the first value.

39. The electronics module of claim 38, wherein the contextual information comprises force data indicative of a force applied to the electronics module.

40. The electronics module of claim 39, wherein the force data indicates that the electrical sensor is connected to an electrode of the wearable article when the force data has a first value, and wherein the force data indicates that the optical sensor has line of sight with the skin surface of the user when the force data has a second value different to the first value.

41. The electronics module of claim 40, wherein the contextual information comprises an optical measurement performed by the optical sensor.

42. The electronics module of claim 41, wherein the optical measurement indicates that the electrical sensor is connected to an electrode of the wearable article when the optical measurement has a first value, and wherein the optical measurement indicates that the optical sensor has line of sight with the skin surface of the user when the optical measurement has a second value different to the first value.

43. The electronics module of claim 42, wherein the contextual information comprises magnetic field data measured by a magnetic field sensor of the electronics module.

44. The electronics module of claim 43, wherein the magnetic field data indicates that the electrical sensor is connected to an electrode of the wearable article when the magnetic field data has a first value, and wherein the magnetic field data indicates that the optical sensor has line of sight with the skin surface of the user when the magnetic field data has a second value different to the first value; wherein the electrical sensor comprises a bioelectrical and/or bioimpedance sensor; wherein the optical sensor comprises a PPG sensor.

45. A method performed by an electronics module for monitoring physical activity of a user, the method comprising:
obtaining contextual information from one or more sensors of the electronics module when the electronics module is coupled to a wearable article, the contextual information is indicative of whether the electrical sensor is connected to an electrode of the wearable article and whether the optical sensor has line of sight with a skin surface of the user; and
using the contextual information to automatically configure the operation of an electrical sensor and an optical sensor of the electronics module,
wherein when the contextual information indicates that the electrical sensor is connected to the electrode, the method comprises controlling the electrical sensor to perform measurements, and
wherein when the contextual information indicates that the optical sensor has line of sight with a skin surface of the user, the method comprises controlling the optical sensor to perform measurements.

46. An electronics module for monitoring physical activity of a user, the electronics module is arranged to be removably coupled to any of a single wearable article at a plurality of locations or a plurality of different wearable articles, the electronics module comprising:
an optical sensor arranged to perform optical measurements of the user depending on the particular location at which the electronics module is coupled to the single wearable article or the particular wearable article of the plurality of different wearable articles the electronics module is coupled to;
an electrical sensor arranged to perform electrical measurements of the user depending on the particular location at which the electronics module is coupled to the single wearable article or the particular wearable article of the plurality of different wearable articles the electronics module is coupled to;
a controller arranged to obtain contextual information from one or more sensors of the electronics module when the electronics module couples to the single wearable article at one of the plurality of locations or couples to one of the plurality of different wearable articles, the contextual information is indicative of whether the electrical sensor is connected to an electrode of the wearable article and whether the optical sensor has line of sight with a skin surface of the user,
wherein, when the contextual information indicates that the electrical sensor is connected to the electrode, the controller is arranged to control the electrical sensor to perform measurements, and
wherein, when the contextual information indicates that the optical sensor has line of sight with a skin surface of the user, the controller is arranged to control the optical sensor to perform measurements.
